(19) United States
(12) Patent Application Publication
(10) Pub. No.: US 2026/0315526 A1
(43) Pub. Date: Oct. 15, 2026

(54) A NOVEL FEED COMPOSITION

(71) Applicant: DSM IP Assets B.V., Maastricht (NL)

(72) Inventor: Mathias SPIEGELHOFER, Kaiseraugst (CH)

(21) Appl. No.: 19/483,756

(22) PCT Filed: May 2, 2024
(86) PCT No.: PCT/EP2024/062017

§ 371 Date: Nov. 12, 2025

(30) Foreign Application Priority Data
May 17, 2023 (EP) 23173812.1

(51) Int. Cl. A61K 33/42; A61K 31/19; A61K 31/192; A61P 3/00

(52) U.S. Cl. CPC A61K 33/42; A61K 31/19; A61K 31/192; A61P 3/00

(57) ABSTRACT

The present invention provides a feed composition comprising a) at least one organic acid and/or at least one salt thereof; and b) hypophosphorous acid and/or at least one salt thereof, and use thereof for improving growth performance and/or immunity, and/or reducing mortality of animals.

### TECHNICAL FIELD

The present invention relates to a novel feed composition and use thereof.

### BACKGROUND OF THE INVENTION

Acidifier products including organic acids may be used to maintain food and feed hygiene and prevent spoilage by microorganisms such as bacteria or molds. Organic acids such as benzoic acid, formic acid, propionic acid and acetic acid may be used to control microbial growth in foodstuff, food or feed with the aim to minimize the risk for foodborne diseases. To this end, acidifier products may be added pre- and/or post-production of finished feed. Additionally, acidifier products may be used to improve feed conversion rate and weight gain of animals upon ingestion.

It has now been found surprisingly that organic acids and/or salts thereof in combination with hypophosphorous acid and/or salts thereof provide additional benefits in animals.

### SUMMARY OF THE INVENTION

The present invention provides a feed composition comprising a) at least one organic acid and/or at least one salt thereof; and b) hypophosphorous acid and/or at least one salt thereof.

The present invention also provides a method for improving growth performance and/or immunity, and/or reducing mortality in an animal, comprising administering to the animal the feed composition according to the present invention.

### DESCRIPTION OF THE FIGURES

FIG. 1: Boxplots of hematology parameters after 56 days of feeding the experimental diets (before challenge) and Day 7 after bacterial challenge per tank across the different groups. The box represents the interquartile range (IQR: 50% of data are found between Q1 to Q3). A line across the box indicates the median. The lines/whiskers outside the box extend by Q1-1.5×IQR (25% of data) and Q3+1.5×IQR (25% of data), respectively. Outliers outside the whiskers are represented by individual marks. The darkblack rhombus indicates the mean. Group 1=control diet, group 2=Biotronic PX Top3, group 3=MR.

### DETAILED DESCRIPTION OF THE INVENTION

Animals: The term “animal” or “animals” refers to any animal except human. Examples of the animals include but are not limited to pigs or swine such as piglets, growing pigs and sows; poultry such as turkeys, ducks, quail, guinea fowl, geese, pigeons (including squabs) and chicken (including but not limited to broilers, chicks and layers); pets such as cats and dogs; horses; crustaceans such as shrimps and prawns; fish such as amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish. Preferably, the animal is selected from the group consisting of pigs or swine (including but not limited to piglets, growing pigs and sows); poultry such as turkeys, ducks, quail, guinea fowl, geese, pigeons (including squabs) and chicken (including but not limited to broilers, chicks, layers); pets such as cats and dogs; and crustaceans such as shrimps and prawns.

Animal Feed: the term “animal feed” refers to any compound, preparation, or mixture suitable for or intended for intake by an animal and capable of maintaining life and/or promoting production of the animal without any additional substance being consumed except water.

Animal Feed Additive: the term “Animal feed additive” refers to an ingredient or combination of ingredients added to the animal feed, usually used in micro quantities and requires careful handling and mixing. Such ingredient includes but is not limited to vitamins, amino acids, minerals, enzymes, eubiotics, colouring agents, growth improving additives and I aroma compounds/flavourings, polyunsaturated fatty acids (PUFAS); reactive oxygen generating species, antioxidants, anti-microbial peptides, anti-fungal polypeptides and mycotoxin management compounds etc.,

### Feed Composition Comprising Organic Acid(s) and Hypophosphorous Acid

In the first aspect, the present invention provides a feed composition comprising a) at least one organic acid and/or at least one salt thereof; and b) hypophosphorous acid and/or at least one salt thereof.

In the present invention, the at least one organic acid may be selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and keto carboxylic acids, and/or at least one salt thereof. Merely for clarification, examples of the short monocarboxylic acids having between 1 and 6 carbon atom(s) include but are not limited to formic acid, acetic acid, propionic acid, butyric acid, isobutyric acid, 3-methylbutyric acid, 2-methylbutyric acid, 2-ethylbutyric acid, valeric acid, hexanoic acid. Examples of the saturated dicarboxylic acids include but are not limited to adipic acid and succinic acid. An example of the unsaturated dicarboxylic acid is fumaric acid. Examples of the unsaturated carboxylic acids include but are not limited to sorbic acid and oleic acid. Examples of the saturated carboxylic acids include but are not limited to stearic acid, octanoic acid (also referred to as caprylic acid), decanoic acid (also referred to as capric acid) and dodecanoic acid (also referred to as lauric acid). Examples of the hydroxylcarboxylic acids include but are not limited to lactic acid, malic acid (D-, or L-, or D/L-malic acid), citric acid and tartaric acid. Examples of the aromatic carboxylic acids include but are not limited to benzoic acid and cinnamic acid. An example of keto carboxylic acid is pyruvic acid.

Preferably, the at least one organic acid may be selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid and gallic acid, or at least one salt thereof.

More preferably, the at least one organic acid may be selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid and pyruvic acid, or at least one salt thereof.

Further preferably, the at least one organic acid may be selected from the group consisting of acetic acid, butyric acid, citric acid, formic acid, fumaric acid, lactic acid, octanoic acid, propionic acid, pyruvic acid, benzoic acid, sorbic acid, succinic acid and valeric acid, or at least one salt thereof.

In the present invention, the at least one salt of the organic acid may be any one of metal salts such as potassium, sodium or calcium salts, and ammonium salts. Examples of the salts of the organic acid include but are not limited to ammonium formate, potassium diformate, sodium diacetate, calcium acetate, ammonium propionate, sodium propionate, calcium propionate, calcium lactate, potassium sorbate, sodium formate, calcium formate, sodium butyrate, sodium sorbate, potassium citrate, sodium citrate, calcium citrate, and benzoates such as sodium benzoate, magnesium benzoate, manganese benzoate, potassium benzoate, aluminium benzoate, calcium benzoate and ferric benzoate.

Examples of commercial organic acid products are VevoVitall® (DSM Nutritional Products, Switzerland), Biotronic® (DSM Nutritional Products, Austria), Amasil®, Luprisil®, Lupro-Grain®, Lupro-Cid®, Lupro-Mix® (BASF), n-Butyric Acid AF (OXEA) and Adimix Precision (Nutriad).

In the present invention, the at least one salt of hypophosphorous acid may be selected from the group consisting of sodium hypophosphite, magnesium hypophosphite, manganese hypophosphite, potassium hypophosphite, aluminum hypophosphite, calcium hypophosphite, ammonium hypophosphite and ferric hypophosphite, preferably sodium hypophosphite, magnesium hypophosphite, manganese hypophosphite and potassium hypophosphite, more preferably sodium hypophosphite.

In the present invention, the feed composition according to the invention may comprise more than one organic acid and/or the at least one salt thereof and hypophosphorous acid and/or at least one salt thereof. For example, such a feed composition may comprise two, three, four or even more organic acids in combination with hypophosphorous acid and/or at least one salt thereof. In an example, the feed composition comprises at least three organic acids, especially formic acid, acetic acid, propionic acid, and a salt of hypophosphorous acid selected from the group consisting of sodium hypophosphite, manganese hypophosphite, magnesium hypophosphite and potassium hypophosphite, preferably sodium hypophosphite and manganese hypophosphite, more preferably sodium hypophosphite. In another example, the feed composition comprises at least four organic acids, especially formic acid, acetic acid, propionic acid, benzoic acid, and a salt of hypophosphorous acid selected from the group consisting of sodium hypophosphite, manganese hypophosphite, magnesium hypophosphite and potassium hypophosphite, preferably sodium hypophosphite and manganese hypophosphite, more preferably sodium hypophosphite.

In the present invention, the feed composition may be provided in a particular manner, wherein the molar ratio of the at least one organic acid and/or the at least one salt thereof to hypophosphorous acid and/or the at least one salt thereof is from 100:0.1 to 0.1:100, preferably from 100:1 to 1:100, more preferably from 50:1 to 1:50, even more preferably from 20:1 to 1:20, and the most preferably from 10:1 to 1:10.

Such a feed composition according to the present invention may be provided in a form wherein one or more or all of the components is/are provided in solid form (e.g. salt, powder, granulate, pellet etc.) or in liquid form (e.g. aqueous, gel, viscous). It is also considered that the feed composition may be provided in a manner, wherein one component (e.g. one or more organic acid(s) is provided in liquid form and a second component is provided in solid form (e.g. hypophosphorous acid), and the composition is formed by combining the two components in a mixture or in a separate form.

As anticipated by any person skilled in the art, the feed composition according to the present invention may be formulated as an animal feed additive. Accordingly, the feed composition of the present invention may also include micro-ingredients.

The micro-ingredients include but are not limited to aroma compounds; antimicrobial peptides; polyunsaturated fatty acids (PUFAs); reactive oxygen generating species; at least one enzyme, and fat- and water-soluble vitamins, as well as minerals.

Examples of antimicrobial peptides (AMP's) are CAP18, leucocin A, protegrin-1, thanatin, defensin, lactoferrin, lactoferricin, and ovispirin such as novispirin (Robert Lehrer, 2000), plectasins, and statins.

Examples of polyunsaturated fatty acids are C₁₈₋, C₂₀₋ and C₂₂₋polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Examples of enzyme are phytase (EC 3.1.3.8 or 3.1.3.26), galactanase (EC 3.2.1.89), alpha-galactosidase (EC 3.2.1.22), phospholipase A 1 (EC 3.1.1.32), phospholipase A2 (EC 3.1.1.4), lysophospholipase (EC 3.1.1.5), phospholipase C (EC 3.1.4.3), and/or phospholipase D (EC 3.1.4.4).

Examples of fat-soluble vitamins include but are not limited to vitamin A, vitamin D3, and vitamin K, e.g. vitamin K₃.

Examples of water-soluble vitamins include but are not limited to vitamin B₁₂, biotin and choline, vitamin B₁, vitamin B₂, vitamin B₆, niacin, folic acid and panthothenate, e.g. Ca-D-panthothenate.

Examples of minerals include but are not limited to calcium, phosphorus, sodium, potassium, magnesium, chlorine, iodine, iron, manganese, copper, molybdenum, cobalt and zinc. Common mineral supplements in feed include but are not limited to limestone, Bone meal, oyster shell, sodium chloride, dicalcium phosphate, manganese sulphate, potassium iodide, and superphosphate. Sources of minerals include meat scraps, fish meal, milk products, ground limestone (calcium), ground oyster shells (calcium), dicalcium phosphate (calcium, phosphorus), defluorinated rock phosphate (phosphorus, calcium), steamed bone meal (phosphorus, calcium), salt (sodium, chlorine, iodine), manganese sulfate (manganese), manganese oxide (manganese), zinc carbonate (zinc), zinc oxide (zinc).

As also anticipated by any person skilled in the art, the feed composition according to unie present invention may further be formulated as an animal feed. Accordingly, the feed composition of the present invention may further include any number of components typical for an animal feed, such as proteins, carbohydrates as defined above, fats and additional additives.

Examples of suitable types of proteins that can be included in the feed include, but are not limited to, meat scraps (lysine), fish meal (lysine, methionine), poultry by-product meal (tryptophan, lysine), blood meal, liver and glandular meal, feather meal (hydrolyzed), animal tankage, milk products, cottonseed meal, peanut meal, soybean meal, sesame meal, sunflower seed meal.

Most feed ingredients (maize, barley, safflower, milo, wheat, rice, bran, etc.) contain approximately 2-5% fat and linoleic acid. Sources of fats include animal tallow (beef), lard, corn oil, and other vegetable oils.

Additional additives include but are not limited to minerals as defined above; antioxidants like BHT (Butylated hydroxytoluene), santoquin, ethoxyquin, butylated hydroxyanisode and diphenyl paraphenyl diamine; pellet binders such as sodium bentonite (clay), liquid or solid by-products of the wood pulp industry, molasses, and guarmeal; coloring agents such as xanthophylls, synthetic carotinoid, and canthaxanthin; probiotics such as strains of lactobacillus and streptococcus; and/or antibiotics such as penicillin, streptomycin, tetracyclines, and aureomycin.

In the present invention, the at least one organic acid and/or the at least one salt thereof may be provided at a concentration of from 0.001% to 10%, preferably from 0.01% to 5%, more preferably from 0.05% to 1% by weight such as 0.05%, 0.1%, 0.2%, 0.3%, 0.4% and 0.5% by weight of the animal feed.

In the present invention, hypophosphorous acid and/or the at least one salt thereof may be provided at a concentration of from 0.0005% to 1%, preferably from 0.001% to 0.5%, more preferably from 0.002% to 0.2%, even more preferably 0.0025% to 0.1% by weight such as 0.0025%, 0.003%, 0.004%, 0.005%, 0.01%, 0.015%, 0.02%, 0.025%, 0.03%, 0.04%, 0.05%, 0.06%, 0.08% and 0.1% by weight of the animal feed.

Method for Improving Performance and/or Immunity, and/or Reducing Mortality of Animals

Surprisingly it has been discovered that the feed composition comprising a) at least one organic acid and/or at least one salt thereof and b) hypophosphorous acid and/or at least one salt thereof according to the present invention provides synergistic effect in improving performance and/or immunity, and/or reducing mortality of animals.

Accordingly, in the second aspect, the present invention provides a method for improving grown performance and/or immunity, and/or reducing mortality in animals, comprising administering to the animals the feed composition, the animal feed additive or the animal feed according to the present invention as described herein.

The present invention also provides use of the feed composition of the present application as described herein in the preparation of an animal feed additive or an animal feed for improving growth performance and/or immunity, and/or reducing mortality in animals.

In the present invention, the growth performance of animals may be characterized or represented by Weight Gain (WG), average weight gain and/or Feed Conversion Ratio (FCR) of the animals.

In the present invention, the immunity of animals may be characterized or represented by total hemocyte count (THC), as well as the amount and percentage of hyaline cells (HCs), semi granular cells (SCs) and/or granular cells (GCs) of hemolymph of the animals.

In the present invention, the mortality of animals may be characterized or represented by percentage of death or survival of animals after challenge according to the present invention.

In the present invention, the improvement is compared to an animal feed additive wherein the at least one organic acid and/or at least one salt thereof and hypophosphorous acid and/or at least one salt thereof are not included (herein referred to as the control). Preferably, one or more of the parameters on performance, immunity and/or mortality of animals is changed in a desired direction by at least 0.5%, such as by at least 0.6%, at least 0.7%, at least 0.8%, at least 0.9%, at least 1.0%, at least 1.2% or at least 1.4%, compared to the control.

In method of the present invention, the at least one organic acid and/or the at least one salt thereof may be administered at a dosage of from 0.001% to 10%, preferably from 0.01% to 5%, more preferably from 0.05% to 1% by weight such as 0.05%, 0.1%, 0.2%, 0.3%, 0.4% and 0.5% by weight of the animal feed.

In the method of the present invention, hypophosphorous acid and/or the at least one salt thereof may be administered at a dosage of from 0.0005% to 1%, preferably from 0.001% to 0.5%, more preferably from 0.002% to 0.2%, even more preferably 0.0025% to 0.1% by weight such as 0.0025%, 0.003%, 0.004%, 0.005%, 0.01%, 0.015%, 0.02%, 0.025%, 0.03%, 0.04%, 0.05%, 0.06%, 0.08% and 0.1% by weight of the animal feed.

The present invention is further illustrated by the following examples.

### EXAMPLES

### Example 1

### Animals and Housing

The trial was performed at the Aquaculture Center for Applied Nutrition (ACAN), Nong Lam University, Linh Trung Ward, Thu Duc District, Ho Chi Minh City, Vietnam from 1 Aug. 2022 until 10 Oct. 2022.

In total, 5000 post-larvae (PL 10) white leg shrimp Litopenaeus vannamei (Specific Pathogen Free) were bought from a local hatchery in Ninh Thuan province (www.shrimpvet.com). At the ACAN facilities, the PL were reared in 2000 L quarantine tanks under recirculating seawater conditions (at 15 ppt salinity, 29-31° C. and pH 8.0-8.2) for 42 days to reach average size at 2.40 g body weight/shrimp. During the rearing period, the shrimp PL were fed with commercial feed adequate for the shrimp.

### Feeding and Treatments

After the rearing period in the quarantine tanks, shrimp that showed normal feeding behavior and no apparent sign of disease were selected for the performance trial. The three groups (six replicates per group, 20 shrimp per replicate) received the basal diet without the supplementation (control group) or with the supplementation of the feed additives Biotronic PX Top 3 (DSM Nutritional Products, Austria) or MR (95 wt % Biotronic PX Top 3 (DSM Nutritional Products, Austria) and 5 wt % Sodium hypophosphite mono-hydrate (NaH2PO2*H2O)) as shown in Table 1. The experimental diet was offered during a period of 70 days (included performance trial and challenge trial).

TABLE 1
Overview of the experimental groups in the performance trial

| Group code | Description |
|---|---|
| 1 | Control diet |
| 2 | Control diet + Biotronic PX Top3 (0.5 kg/ton feed) |
| 3 | Control diet + MR (0.5 kg/ton feed) |

The composition of the basal diet (control diet) used in the trial is shown in Table 2. The experimental diets (group 2-3) were prepared by mixing in the feed additives Biotronic PX Top 3 or MR into the basal feed.

TABLE 2
Formulation of the basal diet

| Description | % |
|---|---|
| local fish meal 60% | 24.00 |
| krill meal | 8.00 |
| soybean meal 46% CP | 30.50 |
| fish oil ACAN | 2.50 |
| liquid lecithin BIO | 1.00 |
| whole wheat | 29.00 |
| Biomin shrimp premix BA192 | 1.00 |
| limestone | 0.45 |
| salt | 0.50 |
| lysine HCL | 0.30 |
| methionine | 0.08 |
| cholesterol | 0.08 |
| choline Chloride 50% | 0.33 |
| threonine | 0.20 |
| vitamin C | 0.06 |
| monocalcium phosphate | 2.00 |
| | 100.00 |

During the entire experiment, water quality was maintained in suitable conditions for shrimp rearing. After completing all sampling for the performance trial, 13 shrimp were randomly selected from each tank and transferred in the challenge tanks (3 groups with 4 replicates per group).

The animals were acclimatized for a period of 3 days in the challenge tanks to allow them to adapt to the new conditions as well as ensure that no mortality occurred after the transfer. Shrimp were challenged with 24 h immersion in a 20-ppm ammonia solution, followed by 1 hour of immersion in 1.12×10⁶ CFU/mL V. parahaemolyticus. After 1 hours of bacterial immersion, 50% of the water from each challenge tank was exchanged with fresh salt water to reduce the bacterial concentration to 50% in each tank.

Shrimp mortality was observed at least twice daily and recorded until the challenge end. Moribund or dead animals were removed. Death shrimp during the first three days after bacterial challenge in good condition were send to lab for bacterial isolation to confirm successful challenge. At the end of the challenge trial, all remaining shrimp were sacrificed, disinfected with chlorine and discarded.

### Experimental Parameters and Analyses

### 1. Growth Performance Parameters

Clinical observations, water quality parameters and mortality were recorded daily. For practical reasons, the animals within each tank were weighed together to record the sum of their weight on day 0, 28 and 56. The mean body weight of the individuals within each tank was calculated by dividing the combined weight of the animals by the number of animals.

Feed consumption was determined for the periods between each weighing day. Feed conversion ratio (FCR) was calculated as the proportion of the total tank-based feed consumption to the weight gain per tank as well as the calculated mean feed consumption per animals and weight gain per animal.

### 2. Immunology Analysis

The sample of hemolymph from 3 shrimps per tank were collected for immunological analysis on the following time points:
1. Sampling at the end of performance trial
2. Sampling: during challenge trial (1 week after challenge)

The animals were randomly selected to analyze the total hemocyte count (THC), as well as the amount and percentage of hyaline cells (HCs), semi granular cells (SCs) and granular cells (GCs).

### 3. Sampling of Animals for EMS Testing During Challenge Trial

Prior to the challenge, a pooled sample containing of four shrimp from the different performance tanks per groups was taken. Additionally, to determine the cause of death after the bacterial challenge, 3-4 dead shrimp in good condition per groups were sent to the lab for Early Mortality Syndrome (EMS/AHPND) testing via real-time PCR at day 5 after the bacterial challenge was done.

### Results and Discussion

### 1. Growth Performance

As shown in Table 3, compare with the control group and the Biotronic PX Top3 group, the mean body weight and the mean body weight gain of the shrimps per tank were higher in group supplemented with the MR.

TABLE 3
Descriptive statistics of growth performance parameters per group

| Day | Group | Mean |
|---|---|---|
| Mean body weight per | 1 | 12.42^{a} |
| tank on d 56 (g) | 2 | 12.98^{ab} |
| | 3 | 13.08^{ab} |
| Mean body weight gain | 1 | 9.76^{ac} |
| per tank d 0-d 56 (g) | 2 | 10.49^{bc} |
| | 3 | 10.56^{bc} |

### 2. Immune Parameter

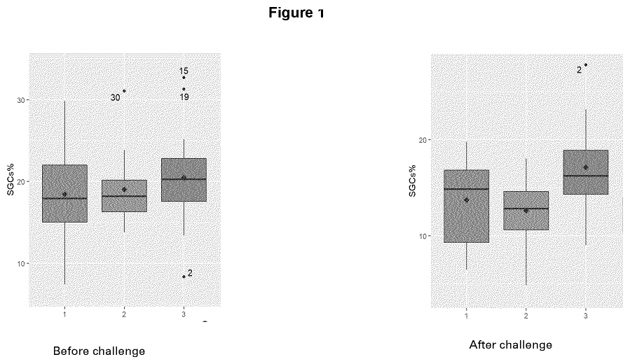

As shown in FIG. 1, compare with the control group and the Biotronic PX Top3 group, the percentage of Small Granular cell (SGC) counts in the group supplemented with the MR was higher before challenge and after challenge.

### 3. Survival in Challenge Trial

The EMS analysis from shrimp samples collected before and after bacterial challenge showed that trial shrimp at the end the performance trial (before challenge) were free of EMS but EMS positive after challenge, indicating the successful challenge with the EMS positive bacteria Vibrio parahaemolyticus.

As shown in Table 4, the survivability during the challenge trial was higher in the group supplemented with the MR, compared with the control group and the Biotronic PX Top3 group during the first week of challenge.

TABLE 4
Survival animal numbers and survival rate after challenge

| | Numbers of alive | Numbers of alive | |
|---|---|---|---|
| | animals at d 0 | animals at d 7 | Survival rate |
| Groups | after challenge | after challenge | after challenge |
| 1 | 60 | 19 | 31.6% |
| 2 | 60 | 21 | 35% |
| 3 | 60 | 26 | 43.3% |

### Conclusion

Feed supplementation with MR improves growth performance and immunity and reduce mortality as compared with the control group.

### Example 2

### Animals and Housing

The trial was carried out in a commercial farm in Lorca (Murcia, Spain). A total of 572 healthy weaning piglets [(Landrace x Large White)×Pietrain], weaned at an average of 26 days of age with initial average body weight of 5.73±0.96 kg, were used for the trial. The environmental conditions were automatically controlled, ensuring an adequate temperature and ventilation for the age of the piglets.

At weaning piglets were classified by body weight into groups of 13 animals (gender mix with the same ratio males/females in each department) and housed in the 44 experimental pens (11 replicates per group). Subsequently they were weighted (individually) and randomly assigned to one of the 4 experimental treatments (CTR, VEV, BIO, SH) based exclusively on body weight.

Although a 2-phase feeding plan (0-14d and 14-38d) was used, the duration of the experimental period was 38 days, distributed in 4 periods: 0-7d, 7-14d, 14-21d and 21-38d. At the end of each period, piglets were weighted (individually do, d7, d14, d21 and d38), and the total feed consumption recorded in order to subsequently calculate the main productive parameters (growth rate, average daily feed consumption and feed conversion rate).

### Experimental Diets

The experimental diets for each of the study phases were formulated using BRILL Software (linear programming method) and FEDNA 2019 composition tables. Same nutrient and ingredient restrictions were used with the only exception of the inclusion of the test products (VEVOVITALL® (DSM Nutritional Products, Switzerland) and BIOTRONIC® TOP 3 (DSM Nutritional Products, Austria) or sodium hypophosphite).

The inclusion levels of the test products as well as the ingredient of the experimental diets are shown in Tables 5-6.

TABLE 5
0-14 d diet composition (%, as fed basis)

| | Groups | | | |
|---|---|---|---|---|
| Ingredient | CTR | VEV | BIO | SH |
| Extruded cereals (corn, rice, oat) | 20.000 | 20.000 | 20.000 | 20.000 |
| Wheat | 20.000 | 19.474 | 19.579 | 19.768 |
| Extruded wheat/Whey powder | 15.000 | 15.000 | 15.000 | 15.000 |
| (82/18) | | | | |
| Soybean meal, 47% CP | 11.828 | 12.076 | 12.027 | 11.938 |
| Corn | 10.028 | 9.764 | 9.816 | 10.032 |
| Barley | 5.241 | 5.103 | 5.131 | 5.180 |
| Whey powder (70/12) | 5.000 | 5.000 | 5.000 | 5.000 |
| Fish meal 68% CP | 3.000 | 3.000 | 3.000 | 3.000 |
| Rovimap CP70¹ | 3.000 | 3.000 | 3.000 | 3.000 |
| Soybean oil | 2.342 | 2.434 | 2.416 | 2.383 |
| Porcine plasma | 1.000 | 1.000 | 1.000 | 1.000 |
| L-Lysine 50 | 0.788 | 0.799 | 0.797 | 0.793 |
| Dextrose | 0.500 | 0.500 | 0.500 | 0.500 |
| Premix vitamin-mineral² | 0.400 | 0.400 | 0.400 | 0.400 |
| Salt | 0.400 | 0.400 | 0.400 | 0.400 |
| Monocalcium phosphate | 0.305 | 0.324 | 0.320 | 0.313 |
| Methionine MHA | 0.274 | 0.281 | 0.279 | 0.277 |
| Calcium carbonate | 0.213 | 0.216 | 0.216 | 0.214 |
| L-Threonine | 0.212 | 0.221 | 0.219 | 0.216 |
| L-Valine | 0.137 | 0.147 | 0.145 | 0.141 |
| Valine 30 - Isoleucine 70 | 0.072 | 0.087 | 0.084 | 0.079 |
| L-Tryptophan | 0.070 | 0.073 | 0.073 | 0.071 |
| L-Histidine | 0.067 | 0.078 | 0.075 | 0.072 |
| Choline Chloride 75 | 0.053 | 0.053 | 0.053 | 0.053 |
| Sweetener | 0.050 | 0.050 | 0.050 | 0.050 |
| Phytase³ | 0.020 | 0.020 | 0.020 | 0.020 |
| VEVOVITALL ® | — | 0.500 | 0.300 | 0.095 |
| BIOTRONIC ® TOP 3 | — | — | 0.100 | — |
| SODIUM HYPOPHOSPHITE | — | — | — | 0.005 |

¹Rovimap CP70: vegetal protein concentrate (70% crude protein)
²Premix vitamin-mineral: 4000000 UI/kg Vit A, 500000 UI/kg Vit D₃, 25000 mg/kg Vit E, 625 mg/kg Vit K₃, 31250 mg/kg Vit C, 45000 mcg/kg biotin, 250 mg/g folic acid, 750 mg/kg Vit B₁, 1750 mg/kg Vit B₂, 1125 mg/kg Vit B₆, 8000 mcg/kg Vit B₁₂, 8000 mg/kg niacinamide, 4500 mg/kg calcium pantothenate, 30000 mg/kg Fe, 13750 mg/kg Cu; 12500 mg/kg Mn, 25000 mg/kg Zn, 250 mg/kg I, 100 mg Se
³Phytase: 6-phytase (OptiPhos ®).

TABLE 6
14-38 d diet composition (%, as fed basis)

| | Groups | | | |
|---|---|---|---|---|
| Ingredient | CTR | VEV | BIO | SH |
| Wheat | 35.000 | 34.560 | 34.648 | 34.807 |
| Barley | 23.892 | 23.592 | 23.652 | 23.760 |
| Soybean meal, 47% CP | 18.000 | 18.250 | 18.200 | 18.110 |
| Corn | 15.028 | 14.840 | 14.878 | 15.064 |
| Soybean oil | 2.930 | 3.020 | 3.002 | 2.970 |
| Rovimap CP70¹ | 1.250 | 1.250 | 1.250 | 1.250 |
| L-Lysine 50 | 0.881 | 0.893 | 0.890 | 0.886 |
| Monocalcium phosphate | 0.713 | 0.732 | 0.728 | 0.722 |
| Calcium carbonate | 0.646 | 0.650 | 0.649 | 0.648 |
| Premix vitamin-mineral² | 0.400 | 0.400 | 0.400 | 0.400 |
| Salt | 0.493 | 0.493 | 0.493 | 0.493 |
| Methionine MHA | 0.230 | 0.236 | 0.235 | 0.233 |
| L-Threonine | 0.223 | 0.232 | 0.230 | 0.227 |
| L-Valine | 0.118 | 0.128 | 0.126 | 0.122 |
| Choline Chloride 75 | 0.053 | 0.053 | 0.053 | 0.053 |
| Sweetener | 0.050 | 0.050 | 0.050 | 0.050 |
| L-Tryptophan | 0.044 | 0.047 | 0.047 | 0.045 |
| Valine 30 - Isoleucine 70 | 0.029 | 0.044 | 0.041 | 0.036 |
| Phytase³ | 0.020 | 0.020 | 0.020 | 0.020 |
| L-Histidine | — | 0.010 | 0.008 | 0.004 |
| VEVOVITALL ® | — | 0.500 | 0.300 | 0.095 |
| BIOTRONIC ® TOP 3 | — | — | 0.100 | — |
| SODIUM HYPOPHOSPHITE | — | — | — | 0.005 |

¹Rovimap CP70: vegetal protein concentrate (70% crude protein)
²Premix vitamin-mineral: 4000000 UI/kg Vit A, 500000 UI/kg Vit D₃, 25000 mg/kg Vit E, 625 mg/kg Vit K3, 31250 mg/kg Vit C, 45000 mcg/kg biotin, 250 mg/g folic acid, 750 mg/kg Vit B₁, 1750 mg/kg Vit B₂, 1125 mg/kg Vit B₆, 8000 mcg/kg Vit B₁₂, 8000 mg/kg niacinamide, 4500 mg/kg calcium pantothenate, 30000 mg/kg Fe, 13750 mg/kg Cu; 12500 mg/kg Mn, 25000 mg/kg Zn, 250 mg/kg I, 100 mg Se
³Phytase: 6-phytase (OptiPhos ®).

### Experimental Parameters and Analyses

Average body weight at d0, d7, d14, d21 and d38, kg/piglet
Average daily weight gain (ADWG) 0-7, 7-14, 14-21, 21-38, 0-14, 14-38, and 0-38d, kg/day
Average daily feed intake (ADFI) 0-7, 7-14, 14-21, 21-38, 0-14, 14-38, and 0-38d, kg/day
Feed conversion ratio (FCR) 0-7, 7-14, 14-21, 21-38, 0-14, 14-38, and 0-38d, kg/kg
Withdrawn ratio 0-7, 7-14, 14-21, 21-40, 0-14, 14-38, and 0-38d, %.
Mortality rate 0-7, 7-14, 14-21, 21-40, 0-14, 14-38, and 0-38d, %

### Results and Discussion

As shown in Table 7, compared with the control group and the organic acids group (VEV and BIO), the average daily weight gain was highest and the feed conversion ratio (FCR) is lowest in the SH group which were supplemented with VevoVitall® and sodium hypophosphite. In addition, the SH group provided much lower withdrawn ratio and mortality ratio of piglets.

TABLE 7
Piglet productive performance 0-7 d post-weaning

| | CTR | VEV | BIO | SH |
|---|---|---|---|---|
| Animal number | 11 | 11 | 11 | 11 |
| Body weight d 0 (kg/piglet) | 5.734 | 5.730 | 5.730 | 5.739 |
| Body weight d 7 (kg/piglet) | 6.273 | 6.169 | 6.231 | 6.290 |
| Average daily weight gain (kg/d) | 0.077 | 0.062 | 0.071 | 0.080 |
| Average daily feed intake (kg/d) | 0.156 | 0.144 | 0.159 | 0.157 |
| Feed conversion ratio (kg/kg) | 2.545 | 2.943 | 2.889 | 2.153 |
| Withdrawn ratio (%) | 0.70 | 1.40 | 1.40 | 0.00 |
| Mortality rate (%) | 2.80 | 2.10 | 2.80 | 0.70 |

### Conclusion

The combination of organic acids and sodium hypophosphite improved growth performance and reduced mortality rate in the Pre-Starter period of piglets (0-7d post-weaning).

1. A feed composition comprising a) at least one organic acid and/or at least one salt thereof; and b) hypophosphorous acid and/or at least one salt thereof.

2. The feed composition of claim 1, wherein the at least one organic acid is selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids and keto carboxylic acids.

3. The feed composition of claim 1, wherein the at least one organic acid is selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid and gallic acid, or at least one salt thereof.

4. The feed composition of claim 1, wherein the at least one organic acid is selected from the group consisting of acetic acid, butyric acid, citric acid, formic acid, fumaric acid, lactic acid, octanoic acid, propionic acid, pyruvic acid, benzoic acid, sorbic acid, succinic acid and valeric acid, or at least one salt thereof.

5. The feed composition claim 1, wherein the at least one salt of the organic acid is any one of metal salts such as potassium, sodium or calcium salts, and ammonium salts, including but not limited to ammonium formate, potassium diformate, sodium diacetate, calcium acetate, ammonium propionate, sodium propionate, calcium propionate, calcium lactate, potassium sorbate, sodium formate, calcium formate, sodium butyrate, sodium sorbate, potassium citrate, sodium citrate, calcium citrate, and benzoates such as sodium benzoate, magnesium benzoate, manganese benzoate, potassium benzoate, aluminium benzoate, calcium benzoate and ferric benzoate.

6. The feed composition of claim 1, wherein the at least one salt of hypophosphorous acid is selected from the group consisting of sodium hypophosphite, magnesium hypophosphite, manganese hypophosphite, potassium hypophosphite, aluminum hypophosphite, calcium hypophosphite, ammonium hypophosphite and ferric hypophosphite, preferably sodium hypophosphite, magnesium hypophosphite, manganese hypophosphite and potassium hypophosphite, more preferably sodium hypophosphite.

7. The feed composition of claim 1, wherein wherein the molar ratio of the at least one organic acid and/or the at least one salt thereof to the hypophosphorous acid and/or the at least one salt thereof is from 100:0.1 to 0.1:100, preferably from 100:1 to 1:100, more preferably from 50:1 to 1:50, even more preferably from 20:1 to 1:20, and the most preferably from 10:1 to 1:10.

8. The feed composition of claim 1, wherein the at least one organic acid and/or the at least one salt thereof is provided at a concentration of from 0.001% to 10%, preferably from 0.01% to 5%, more preferably from 0.05% to 1% by weight such as 0.05%, 0.1%, 0.2%, 0.3%, 0.4% and 0.5% by weight of the animal feed.

9. The feed composition of claim 1, wherein hypophosphorous acid and/or the at least one salt thereof is provided at a concentration of from 0.0005% to 1%, preferably from 0.001% to 0.5%, more preferably from 0.002% to 0.2%, even more preferably 0.0025% to 0.1% by weight such as 0.0025%, 0.003%, 0.004%, 0.005%, 0.01%, 0.015%, 0.02%, 0.025%, 0.03%, 0.04%, 0.05%, 0.06%, 0.08% and 0.1% by weight of the animal feed.

10. The feed composition of claim 1, for improving performance and/or immunity, and/or reducing mortality of animals.

11. An animal feed additive or animal feed comprising the feed composition of claim 1.

12. Use of the feed composition according to claim 1 in the preparation of an animal feed additive or animal feed for improving performance and/or immunity, and/or reducing mortality of animals.

13. The use of claim 12, wherein the animal is selected from the group consisting of pigs or swine such as piglets, growing pigs and sows; poultry such as turkeys, ducks, quail, guinea fowl, geese, pigeons (including squabs) and chicken (including but not limited to broilers, chicks and layers); pets such as cats and dogs; horses; crustaceans such as shrimps and prawns; fish such as amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish.

14. A method for improving growth performance and/or immunity, and/or reducing mortality in an animal, comprising administering to the animal the feed composition of claim 1.

15. The method of claim 14, wherein the animal is selected from the group consisting of pigs or swine such as piglets, growing pigs and sows; poultry such as turkeys, ducks, quail, guinea fowl, geese, pigeons (including squabs) and chicken (including but not limited to broilers, chicks and layers); pets such as cats and dogs; horses; crustaceans such as shrimps and prawns; fish such as amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish.
